(19) United States
(12) Patent Application Publication
(10) Pub. No.: US 2020/0367774 A1
(43) Pub. Date: Nov. 26, 2020

(54) NONINVASIVE INTRACRANIAL PRESSURE MEASURING DEVICE

(71) Applicant: AJOU UNIVERSITY INDUSTRY-ACADEMIC COOPERATION FOUNDATION, Suwon-si, Gyeonggi-do (KR)

(72) Inventor: Ji Man HONG, Yongin-si, Gyeonggi-do (KR)

(73) Assignee: AJOU UNIVERSITY INDUSTRY-ACADEMIC COOPERATION FOUNDATION, Suwon-si, Gyeonggi-do (KR)

(21) Appl. No.: 16/957,717

(22) PCT Filed: Dec. 24, 2018
(86) PCT No.: PCT/KR2018/016570

§ 371 Date: Jun. 24, 2020

(30) Foreign Application Priority Data
Dec. 26, 2017 (KR) 10-2017-0179787

(51) Int. Cl. A61B 5/03; A61F 9/02; A61B 8/02; A61B 8/00

(52) U.S. Cl. CPC A61B 5/031; A61F 9/02; A61B 8/02; A61B 8/46; A61B 8/4455; A61B 8/54; A61B 8/4209; A61B 8/4477

(57) ABSTRACT

The present invention is provided with a noninvasive intracranial pressure measuring device for measuring an intracranial pressure of a person, includes goggles worn on the face of the person; a first probe provided on the goggles, and emitting ultrasonic waves to an optic nerve sheath so as to measure a diameter of the optic nerve sheath; a second probe provided on the goggles, and emitting ultrasonic waves to the ophthalmic artery so as to measure a pulsatility index of the ophthalmic artery; and a controller for calculating the intracranial pressure on the basis of measured values received from the first probe and the second probe.

### TECHNICAL FIELD

The present invention relates to a noninvasive intracranial pressure measuring device, and more particularly, to a device for noninvasively measuring an intracranial pressure of a person.

### BACKGROUND

In general, an intracranial pressure measuring device is a device for measuring an intracranial pressure of a person. The brain of a person anatomically exists inside a sturdy bone called a cranium, and is safely protected from an external impact. However, when the brain is severely impacted or damaged, a cerebral edema phenomenon occurs causing the brain to swell. In this state, the brain being swelled by a protection device called the cranium is pressed in the closed cranium, which causes the intracranial pressure to increase, resulting in the death of the patient. Therefore, measuring the intracranial pressure of a person to suppress the sudden increase in the intracranial pressure and taking appropriate medical measures accordingly can be said to be a most neurologically basic matter in treating and managing a critically ill patient.

On the other hand, in the intracranial pressure measuring device, there are an invasive type of inserting a probe into the cranium of a person to measure the intracranial pressure, and a noninvasive type of measuring the intracranial pressure without inserting the probe into the cranium. Since the brain of a person is a very sensitive organ, exposure thereof to an external environment can adversely affect to the patient. Therefore, in measuring the intracranial pressure of a person, it can be seen that the noninvasive intracranial pressure measuring device is used more frequently than the invasive intracranial pressure measuring device.

As related to such a noninvasive intracranial pressure measuring device, Korean Patent Unexamined Publication No. 10-2005-0056100 discloses an ultrasonic cerebral blood flow measuring device using a wireless controller for charging. The cerebral blood flow measuring device of the related art is characterized in that the intracranial pressure is calculated by analyzing a return waveform by emitting ultrasonic waves to the cerebral blood vessels of the middle cerebral artery.

In this case, for the cerebral blood flow measuring device of the related art, a person directly holds the measuring device to come into contact with the head of the patient to measure the intracranial pressure of the patient. Therefore, there is a limit in that the intracranial pressure of the patient has to be calculated on the basis of only information obtained from the cerebral blood vessels. Therefore, according to the cerebral blood flow measuring device of the related art, in a case where an unskilled operator operates the measuring device, a measurement error of the intracranial pressure is large, and even if a skilled operator operates the measuring device, the intracranial pressure is measured on the basis of only the information obtained by measuring the cerebral blood vessels. Therefore, there is a problem that an accurate intracranial pressure value cannot be calculated.

In addition, in a case of a patient 65 years of age or older, there are 30% or more of cases where ultrasonic waves do not pass through the cranium. In a case of a patient whose cranium is destroyed due to the injury of the cranium, there is a limitation in that the intracranial pressure cannot be measured. Therefore, according to the cerebral blood flow measuring device of the related art, there is a problem that the intracranial pressure cannot be measured for all patients.

### SUMMARY OF INVENTION

### Technical Problem

The present invention is created to solve the problems described above, and an object of the present invention is to provide a noninvasive intracranial pressure measuring device that accurately measures an intracranial pressure of a patient regardless of a skill level of an operator, and calculates the intracranial pressure on the basis of various data, thereby improving the accuracy of a value of the calculated intracranial pressure.

### Solution to Problem

The present invention is provided with a noninvasive intracranial pressure measuring device for measuring an intracranial pressure of a person, the device including goggles worn on the face of the person; a first probe provided on the goggles, and emitting ultrasonic waves to an optic nerve sheath so as to measure a diameter of the optic nerve sheath; a second probe provided on the goggles, and emitting ultrasonic waves to the ophthalmic artery so as to measure a pulsatility index of the ophthalmic artery; and a controller for calculating the intracranial pressure on the basis of measured values received from the first probe and the second probe.

The noninvasive intracranial pressure measuring device may further include a third probe provided on the goggles, and emitting ultrasonic waves to the ophthalmic artery and the cranial cavity external artery so as to measure diameters of the ophthalmic artery and the cranial cavity external artery, respectively. The controller may calculate the intracranial pressure on the basis of measured values received from the first to third probes.

The noninvasive intracranial pressure measuring device may further include angle changing means for changing an angle of the first probe. The controller may select a maximum value among the diameters of the optic nerve sheaths received from the first probe to calculate a first intracranial pressure, and calculate a second intracranial pressure on the basis of the measured value received from the second probe.

The noninvasive intracranial pressure measuring device may further include pressing means for applying a pressure to the ocular globe of a person and transmitting information on a pressure value thereof to the controller. The controller may calculate a third intracranial pressure on the basis of the pressure value of the pressing means when the diameter of the ophthalmic artery matches the diameter of the cranial cavity external artery.

The noninvasive intracranial pressure measuring device may further include an intracranial pressure display unit that receives information on an average value of the first to third intracranial pressures from the controller and displays the information to an outside.

The noninvasive intracranial pressure measuring device may further include a probe fixation body provided on the goggles and fixing the first to third probes to the goggles.

### Advantageous Effects

According to the noninvasive intracranial pressure measuring device according to the present invention, the intracranial pressure of the patient can be accurately measured regardless of the skill level of the operator by providing the goggles and the probe fixation body fixed thereto. The first probe, the second probe, and the third probe are provided, and thereby the accuracy of the calculated intracranial pressure value obtained by calculating the intracranial pressure on the basis of various data can be improved.

In addition, according to the noninvasive intracranial pressure measuring device according to the present invention, even in a case where the intracranial arterial blood flow cannot be measured due to the cranium, the flow of blood passing through the intracranial artery can be measured at high resolution by using blood vessels in the bone-free ocular area. In addition, the optic nerve sheath is measured at high resolution, which enables the intracranial pressure to be estimated in the ocular globe, and thereby the intracranial pressure can be measured more accurately.

### BRIEF DESCRIPTION OF THE DRAWINGS

FIG. 1 is a view illustrating a noninvasive intracranial pressure measuring device according to an example of the present invention.

FIG. 2 is a view illustrating a vascular system and a nervous system in the back of the ocular globe.

FIG. 3 is a view illustrating a cross section of the ocular globe.

FIG. 4 is a view illustrating the vascular system inside and outside a cranial cavity.

### DETAILED DESCRIPTION OF THE EMBODIMENTS

The present invention is described with reference to an example illustrated in the drawings, but this is only exemplary, and it will be understood that those skilled in the art can make various modifications and other examples equivalents therefrom. Therefore, the true technical protection scope of the present invention should be defined by the technical spirit of the appended claims.

Hereinafter, a noninvasive intracranial pressure measuring device according to an example of the present invention will be described in detail.

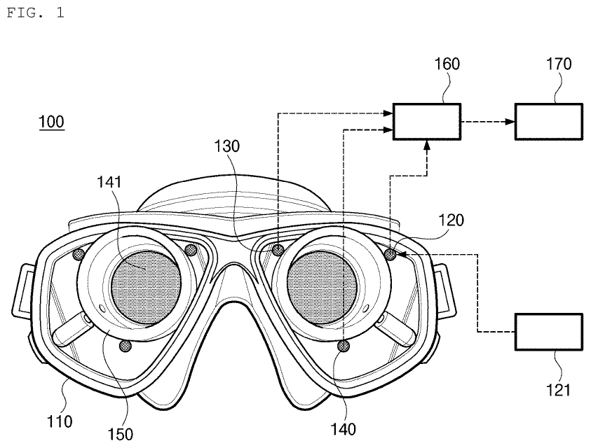

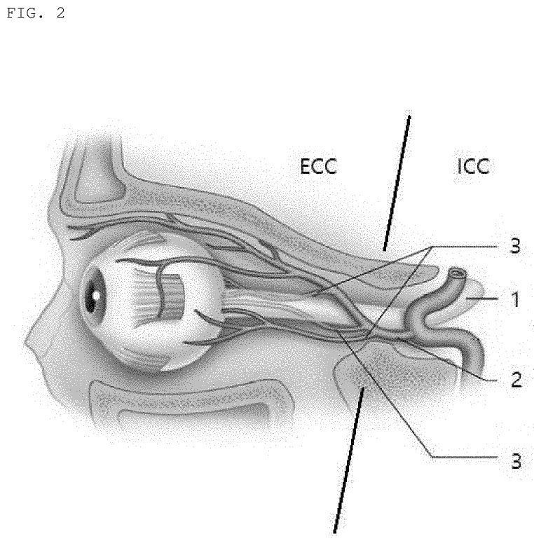

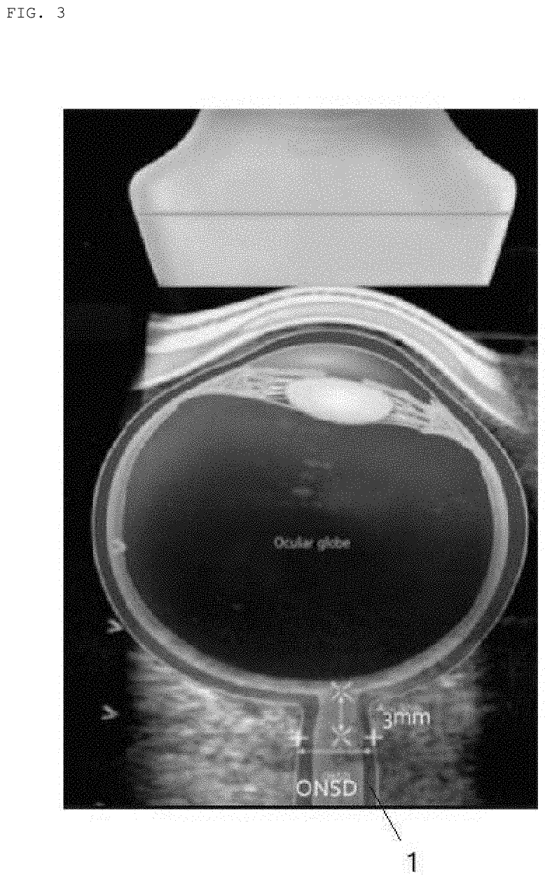

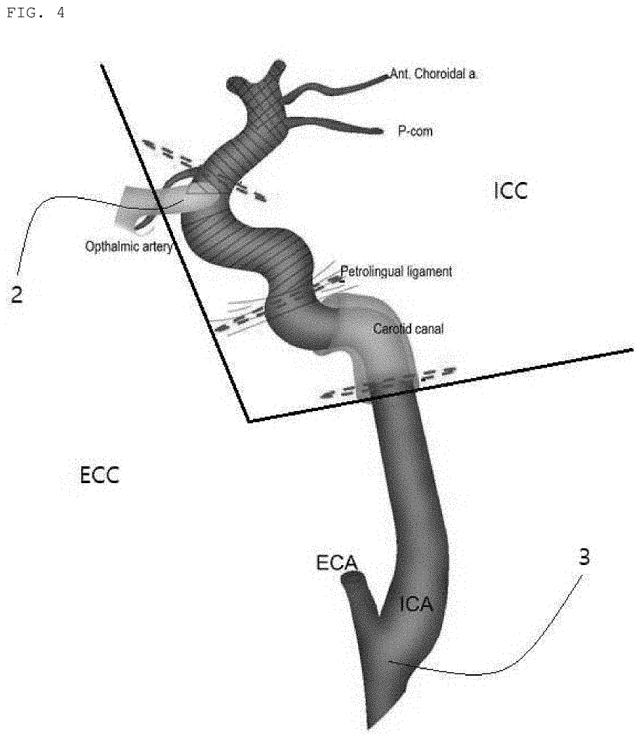

FIG. 1 is a view illustrating the noninvasive intracranial pressure measuring device according to the example of the present invention, FIG. 2 is a view illustrating a vascular system and a nervous system in the back of the ocular globe, FIG. 3 is a view illustrating a cross section of the ocular globe, and FIG. 4 is a view illustrating the vascular system inside and outside the cranial cavity.

Referring to the drawings, a noninvasive intracranial pressure measuring device 100 according to the example of the present invention is a device for noninvasively measuring the intracranial pressure of a person, and includes goggles 110, first to third probes 120, 130, and 140, a probe fixation body 150, a controller 160, and an intracranial pressure display unit 170.

The goggles 110 are provided with the first to third probes 120, 130, and 140, and the probe fixation body 150, and are worn on the face of a person. The first to third probes 120, 130, and 140 can detect noninvasively inside the body of the person.

The first probe 120 emits ultrasonic waves to an optic nerve sheath 1 existing in the back of the ocular globe of the person, and detects ultrasonic waves reflected from the optic nerve sheath 1 to measure a diameter of the optic nerve sheath 1. The frequency of the ultrasonic waves reflected from the optic nerve sheath 1 changes, and the first probe 120 detects the frequency displacement of the ultrasonic waves generated in this way to measure the diameter of the optic nerve sheath 1.

The second probe 130 emits ultrasonic waves to an ophthalmic artery 2 located adjacent to the optic nerve sheath 1, and detects ultrasonic waves reflected from the ophthalmic artery 2 to measure a pulsatility index of the ophthalmic artery 2. The pulsatility index refers to an amount of a change in blood flow speed in a blood vessel, and is a value obtained by dividing a maximum blood flow speed in the blood vessels in a systolic period of the heart and a minimum blood flow speed in the blood vessels in a diastolic period of the heart by an average blood flow speed in the blood vessels during a heartbeat cycle.

In this case, the ultrasonic waves emitted from the second probe 130 may correspond to double B-mode ultrasonic waves. The double B-mode ultrasonic waves refer to a combination of a B-mode (brightness mode) for viewing a structure of the blood vessels and a pulse wave doppler for examining the blood flow speed and a waveform. The B-mode is mainly used to obtain morphological information on a blood vessel wall or a blood vessel lumen, and the pulse wave doppler is mainly used to obtain functional information on the blood flow regarding the speed or direction of the blood flow. Therefore, the double B-mode ultrasonic waves emitted from the second probe 130 may correspond to ultrasonic waves in which the pulse wave doppler appears more dominantly than the B-mode.

The third probe 140 emits the ultrasonic waves into the ophthalmic artery 2 and the cranial cavity external artery 3, respectively, and detects the ultrasonic waves reflected from the ophthalmic artery 2 and the cranial cavity external artery 3 to measure the diameters of the ophthalmic artery 2 and the cranial cavity external artery 3, respectively. Here, the cranial cavity refers to a cavity of the cranium formed to accommodate the brain, and the cranial cavity external artery 3 is distinguished from a cranial cavity internal artery existing in the intracranial cavity (ICC), and refers to the artery existing in the extracranial cavity (ECC). The cranial cavity external artery 3 may correspond to any one selected from an internal carotid artery (ICA), an external carotid artery (ECA), a posterior ciliary artery, a central retinal artery, and the like. In this case, the ultrasonic waves emitted by the third probe 140 may also correspond to the double B-mode ultrasonic waves. However, in this case, the double B-mode ultrasonic waves, unlike the ultrasonic waves emitted from the second probe 130, may correspond to ultrasonic waves in which the B-mode appears more dominantly than the pulsed wave doppler.

The probe fixation body 150 is installed on the goggles 110 and fixes the first to third probes 120, 130, and 140 to the goggles 110. In the related art, the intracranial pressure of a person was measured in such a way that an operator holds the probe and allows the probe to come into direct contact with the body of a person. Therefore, in a case of the related art, there is a problem that the error of the intracranial pressure to be measured is large depending on the skill of the operator. However, in a case of the noninvasive intracranial pressure measuring device 100 according to the example of the present invention, the first to third probes 120, 130, and 140 are fixed to the goggles 110 by the probe fixation body 150, and the goggles 110 are worn directly on the head of the patient. Therefore, the intracranial pressure of the patient can be measured regardless of the holding of the operator. Therefore, according to the noninvasive intracranial pressure measuring device 100 according to the example of the present invention, it is possible to obtain a result of the measurement of the intracranial pressure with a small error regardless of the skill of the medical staff.

The controller 160 is connected to the first to third probes 120, 130, and 140, and receives information on the measured values from the first to third probes 120, 130, and 140. In addition, the controller 160 calculates a first intracranial pressure on the basis of data on the diameter of the optic nerve sheath 1 measured by the first probe 120, calculates a second intracranial pressure on the basis of data on the pulsatility index of the ophthalmic artery 2 measured by the second probe 130, and calculates a third intracranial pressure on the basis of data on the diameters of the ophthalmic artery 2 and the cranial cavity external artery 3 measured by the third probe 140.

In relation to the first intracranial pressure, the noninvasive intracranial pressure measuring device 100 according to the example of the present invention may further include an angle changing unit 121. The angle changing unit 121 automatically and finely converts an angle at which the first probe 120 is disposed and changes an incident angle of the ultrasonic waves emitted from the first probe 120 toward the optic nerve sheath 1. Accordingly, the angle changing unit 121 allows the first probe 120 to measure various diameter values of the optic nerve sheath 1.

The controller 160 selects a largest value among various diameter values of the optic nerve sheath 1 received from the first probe 120. Then, the controller 160 calculates the first intracranial pressure on the basis of the maximum diameter value of the optic nerve sheath 1. As illustrated in FIG. 3, the optic nerve sheath 1 is configured of the optic nerve, which is indicated in yellow in the back of the ocular globe, and the cerebrospinal fluid (CSF) surrounding the optic nerve, which is indicated in black. When brain edema occurs, the proportion of cerebrospinal fluid is increased while the intracranial pressure is increased, and thereby the black area illustrated in FIG. 3 is enlarged. The expansion of the cerebrospinal fluid due to the increase in intracranial pressure occurs within minutes or hours and reflects the increased intracranial pressure. Therefore, it can be said that obtaining the maximum diameter of 3 mm inner part from a border of the retina (surface on the back side of the ocular globe) may be a the standard method of obtaining the diameter of the optic nerve sheath 1, and the first intracranial pressure calculated on the basis of the maximum diameter value of the optic nerve sheath 1 thus obtained reflects the increased intracranial pressure. Therefore, a more accurate intracranial pressure value is exhibited than that measured by other measurement methods.

In relation to the third intracranial pressure, the noninvasive intracranial pressure measuring device 100 according to the example of the present invention may further include a pressing unit 141. The pressing unit 141 applies a pressure to the ocular globe of the person and transmits information on the pressure value to the controller 160. In this case, the pressing unit 141 may apply the pressure to the eyelid of the person or come into direct contact with the ocular globe of the person to directly apply the pressure to the ocular globe. Further, the pressing unit 141 may apply the pressure to the ocular globe of the person by using the pressure of air, or come into mechanical contact with the eyelid or the ocular globe of the person to apply the pressure to the ocular globe of the person.

As described above, in a case where the pressing unit 141 applies the pressure to the ocular globe of the person, the diameter of the ophthalmic artery 2 is changed. The controller 160 detects in real time the diameter of the ophthalmic artery 2 and the diameter of the cranial cavity external artery 3 measured by the third probe 140, and when the diameter of the ophthalmic artery 2 matches the diameter of the cranial cavity external artery 3, the pressure exerted by the pressing unit 141 on the ocular globe of the person is defined as the third intracranial pressure.

The controller 160 calculates an average value of the first to third intracranial pressures obtained as described above and transmits the average value to the intracranial pressure display unit 170. Then, the intracranial pressure display unit 170 allows the average value of the first to third intracranial pressures obtained from the controller 160 to be the intracranial pressure of the corresponding patient and displays the average value to the outside. As such, the noninvasive intracranial pressure measuring device 100 according to the example of the present invention measures different intracranial pressure values, respectively on the basis of different methods and measurement values, and defines the average value of these intracranial pressure values as the intracranial pressure of the corresponding patient, and thereby the accuracy of the calculated intracranial pressure value can be improved.

As described above, according to the noninvasive intracranial pressure measuring device 100 according to the present invention, the goggles 110 and the probe fixation body 150 fixed thereto are provided to accurately measure the intracranial pressure of the patient regardless of the skill level of the operator. The first probe 120, the second probe 130, and the third probe 140 are provided to calculate the intracranial pressure on the basis of various data, thereby improving the accuracy of the calculated intracranial pressure value.

In addition, according to the noninvasive intracranial pressure measuring device according to the present invention, even in a case where the intracranial arterial blood flow cannot be measured due to the cranium, the flow of blood passing through the intracranial artery can be measured at high resolution by using blood vessels in the bone-free ocular area. In addition, the optic nerve sheath is measured at high resolution, which enables the intracranial pressure to be estimated in the ocular globe, and thereby the intracranial pressure can be measured more accurately.

1. A noninvasive intracranial pressure measuring device for measuring an intracranial pressure of a person, the device comprising:
goggles worn on the face of the person;
a first probe provided on the goggles, and emitting ultrasonic waves to an optic nerve sheath so as to measure a diameter of the optic nerve sheath;
a second probe provided on the goggles, and emitting ultrasonic waves to the ophthalmic artery so as to measure a pulsatility index of the ophthalmic artery; and
a controller for calculating the intracranial pressure on the basis of measured values received from the first probe and the second probe.

2. The noninvasive intracranial pressure measuring device according to claim 1, further comprising:
a third probe provided on the goggles, and emitting ultrasonic waves to the ophthalmic artery and the cranial cavity external artery so as to measure diameters of the ophthalmic artery and the cranial cavity external artery, respectively,
wherein the controller calculates the intracranial pressure on the basis of measured values received from the first to third probes.

3. The noninvasive intracranial pressure measuring device according to claim 2, further comprising:
angle changing means for changing an angle of the first probe,
wherein the controller selects a maximum value among the diameters of the optic nerve sheaths received from the first probe to calculate a first intracranial pressure, and calculates a second intracranial pressure on the basis of the measured value received from the second probe.

4. The noninvasive intracranial pressure measuring device according to claim 3, further comprising:
pressing means for applying a pressure to the ocular globe of a person and transmitting information on a pressure value thereof to the controller,
wherein the controller calculates a third intracranial pressure on the basis of the pressure value of the pressing means when the diameter of the ophthalmic artery matches the diameter of the cranial cavity external artery.

5. The noninvasive intracranial pressure measuring device according to claim 4, further comprising:
an intracranial pressure display unit that receives information on an average value of the first to third intracranial pressures from the controller and displays the information to an outside.

6. The noninvasive intracranial pressure measuring device according to claim 2, further comprising:
a probe fixation body provided on the goggles and fixing the first to third probes to the goggles.
